(19) United States
(12) Patent Application Publication
(10) Pub. No.: US 2003/0163084 A1
(43) Pub. Date: Aug. 28, 2003

(54) Creation and agitation of multi-component fluids in injection systems

(75) Inventors: David M. Griffiths, Pittsburgh, PA (US); Walter J. Grumski, Pittsburgh, PA (US); Klaus Tiemann, Bonn (DE)

Correspondence Address: GREGORY L BRADLEY, MEDRAD INC, ONE MEDRAD DRIVE, INDIANOLA, PA, 15051

(73) Assignee: Klaus Tiemann

(21) Appl. No.: 10/325,597

(22) Filed: Dec. 19, 2002

Related U.S. Application Data
(60) Provisional application No. 60/343,079, filed on Dec. 20, 2001.

(51) Int. Cl. A61M 37/00

(52) U.S. Cl. 604/82

(57) ABSTRACT

A system for injecting a multi-component fluid into a patient includes a syringe and at least one agitation element moveable within the syringe to agitate the fluid. The agitation element includes surface structures to create vortices/mixing in the vicinity of the agitation element. An agitation element can also include surface structures to reduce the area of contact between the agitation element and another surface (for example, the interior wall of the syringe). An agitation element may also include a coating that includes at least one component of the multi-component fluid to be released into the fluid. A system for injecting a multi-component fluid into a patient includes a syringe and at least two agitating elements moveable within the syringe to agitate the fluid. A first one of the agitating elements has a density greater than a density of the fluid, and a second on of the agitating elements has a density less than the density of the fluid.

### CROSS REFERENCE TO RELATED APPLICATION

This application claims the benefit of U.S. Provisional Application Serial No. 60/343,079, filed on Dec. 20, 2001, the contents of which are incorporated herein by reference.

### BACKGROUND OF THE INVENTION

The present invention relates generally to creation and maintenance of multi-component fluids, and, especially, to systems, devices and methods for use in connection with the creation and agitation of multi-component fluids to be injected into a

In a number of medical procedures, it is desirable to inject a multi-component injection medium into a patient. An example of such a medical procedure is ultrasound imaging.

Ultrasound imaging creates images of the inside of the human body by broadcasting ultrasonic energy into the body and analyzing the reflected ultrasound energy. Differences in reflected energy (for example amplitude or frequency) appear as differences in gray scale or color on the output images. As with other medical imaging procedures, contrast enhancing fluids (often referred to as contrast media) can be injected into the body to increase the difference in the reflected energy and thereby increase the contrast in the image viewed by the operator.

For ultrasonic imaging, the most common contrast media contain many small bubbles. The difference in density of bubbles when compared to water, and thus their difference in sound transmission, makes small gas bubbles excellent means for scattering ultrasound energy. Small solid particles can also serve to scatter ultrasonic energy. Such particles are typically on the order of 1 to 10 microns (that is, 10⁻⁶ to 10⁻⁵ meters) in diameter. These small particles can pass safely through the vascular bed.

Contrast media suitable for use in ultrasound are supplied in a number of forms. Some of these contrast media are powders to which liquid is added just before use. The powder particles cause a gas bubble to coalesce around them. The powder must be mixed with a liquid, and the mixture must be agitated with just the right amount of vigor to get the optimum creation of bubbles. Another type of contrast medium is a liquid that is agitated vigorously with air. There are no solid particles to act as nuclei, but the liquid is a mixture of several liquid components that make relatively stable small bubbles. A third type of contrast medium uses “hard” spheres filled with a gas. These contrast media are typically supplied as a powder that is mixed with a liquid. The goal is to suspend the spheres in the liquid without breaking them. Even though such spheres have a shell that is hard compared to a liquid, they are very small and relatively fragile. It is also possible for the solid particles themselves to act to scatter ultrasonic energy, but the acoustical properties of the solid spheres are not as different from water as those of a gas, so the difference in reflected energy is not as strong.

After mixing/preparation as described above, the contrast medium is drawn into a syringe or other container for injection into the patient. Typically, the fluid is injected into a vein in the arm of the patient. The blood dilutes and carries the contrast medium throughout the body, including to the area of the body being imaged.

It is becoming more common for a microprocessor controlled power injector to be used for injecting the contrast medium to maintain a consistent flow over a long time, thereby providing a consistent amount of contrast medium (number of particles) in the blood stream. If there are too few particles in a region of interest, for example, there is insufficient image contrast and the diagnosis cannot be made. If too many particles are present, too much energy is reflected, resulting in blooming or saturation of the ultrasound receiver.

Although a power injector can inject contrast medium at a constant flow rate, there must be a constant number of bubbles per volume of fluid injected to provide a constant image contrast. Because a gas is significantly less dense than water and other liquids, however, gas bubbles will rise in a liquid. The rate of rise is related to the diameter of the gas bubble. This density difference is useful to quickly separate large bubbles created during the initial mixing. However, the small bubbles desired for image enhancement will also rise slowly. Solid particles, on the other hand, tend to settle or sink because most solids are more dense than water. Many minutes can elapse between the initial mixing of the contrast medium and the injection into the patient, and/or the injection itself may be several minutes in duration. However, certain multi-component contrast media undergo significant separation after only a few minutes. If the concentration of particles changes over the volume of fluid, the image contrast will degrade.

It is, therefore, very desirable to develop systems, devices and method to maintain multi-component contrast media in a mixed or homogeneous state throughout an injection proceeding.

### SUMMARY OF THE INVENTION

The present invention provides generally, devices, systems and methods for creating and/or agitating a multi-component medium (for example, an ultrasound contrast medium) suitable for injection into a patient.

In one aspect, the present invention provides a system for injecting a multi-component fluid into a patient including a syringe and at least one agitation element moveable within the syringe to agitate the fluid. The agitation element includes surface structures to create mixing in the vicinity of the agitation element. The agitation element can, for example, be adapted to be moved by magnetic force and/or gravitational force.

In one embodiment, the agitation element is generally spherical in shape with channels formed therein. In another embodiment, the agitation element includes a base that is generally spherical in shape and has a mesh overlain thereon. The agitation element can also be a generally hollow mesh or wire frame structure (for example, generally spherical in shape).

Preferably, the size of the agitating element, the size of the surface structures and the velocity with which the agitating element is moved within the fluid are adapted to create vortices within the fluid without creating turbulent flow of a magnitude to damage a significant number of ultrasound scattering particles disposed within the fluid. For example, Kármán vortex streets can be formed.

The present invention also provides a system for injecting a multi-component fluid into a patient including a syringe and at least one agitation element moveable within the syringe to agitate the fluid. The agitation element includes surface structures to reduce the area of contact between the agitation element and another surface (for example, the syringe wall). Reducing the contact between the agitation element and the syringe wall prevents the destruction of, for example, ultrasound scattering particles disposed within the fluid. The agitation element can, for example, include projections extending from the surface thereof. The agitation element can be generally spherical in shape.

In another aspect, the present invention provides a system for injecting a multi-component fluid into a patient including a syringe and at least one agitation element moveable within the syringe to agitate the fluid. The agitation element includes a coating that includes at least one component of the multi-component fluid, the coating releasing the component into the fluid. The coating can, for example, be a powder adapted to disperse particles within the fluid. The particles can, for example, be ultrasound bubbles or microspheres or a therapeutic drug.

In still another aspect, the present invention provides a system for injecting a multi-component fluid into a patient including a syringe and at least two agitating elements moveable within the syringe to agitate the fluid. A first one of the agitating elements has a density greater than a density of the fluid, and a second on of the agitating elements has a density less than the density of the fluid. The system preferably further includes a mechanism to impart motion to the syringe to change the orientation of the syringe relative to the orientation of gravitational force.

### BRIEF DESCRIPTION OF THE DRAWINGS

FIG. 1 illustrates a side view of one embodiment of an agitation system of the present invention in which an agitation element is moved within a fluid via magnetic force.

FIG. 2 illustrates a side view of another embodiment of an agitation system of the present invention in which an agitation element is moved within a fluid via magnetic force.

FIG. 3 illustrates a side view of an embodiment of an agitation system of the present invention in which an agitation element is moved within a fluid via magnetic force and gravitational force.

FIG. 4 illustrates flow of an element through a fluid in a manner to create Kármán vortex streets.

FIG. 5 illustrates flow of an element through a fluid in a manner to create mixing flow with an agitated boundary layer.

FIG. 6 illustrates an embodiment of a generally spherical agitation element having surface structure to induce or enhance mixing in the fluid.

FIG. 7 illustrates another embodiment of a generally spherical agitation element having surface structure to induce or enhance mixing in the fluid.

FIG. 8 illustrates an embodiment of a generally spherical agitation element having surface structure to induce or enhance mixing in the fluid and to prevent bubble destruction.

FIG. 9 illustrates an embodiment of a generally spherical element having a surface coated with a substance suitable to produce a multi-component fluid when contacted with a liquid.

### DETAILED DESCRIPTION OF THE INVENTION

In several embodiments, the present invention provides devices, systems and methods to facilitate or to improve the initial creation and/or mixing of an ultrasound contrast medium and to agitate the contrast medium to maintain a relatively uniform distribution of the contrast enhancing agent or particles throughout the liquid contrast medium prior to and/or during an injection procedure. The present invention is, additionally, applicable generally to multi-component fluids wherein the fluid components are not totally miscible and there is a tendency for the components to separate over time (for example, because of differences in density). The present invention is also applicable to miscible or dissolvable materials during the initial preparation phase when a uniform mixture has not yet been created.

In general, the agitation mechanisms of the present invention agitate the contrast medium within a storage volume, container or syringe by movement of one or more agitation elements or members within the contrast medium or fluid.

In several aspects of the present invention, a magnetic field is used to move one or more magnetic or ferromagnetic agitation elements within a syringe. For example, the magnetic field is operable to attract or repel an agitation element disposed within a syringe to move the agitation element within the injection fluid. The agitation element(s) can, for example, include one or more ferromagnetic or magnetic elements such as balls or spheres. Preferably, the movement of the agitation element(s) within the syringe is controlled in a manner that the contrast fluid is maintained is a generally homogeneous state. Likewise, agitation is preferably controlled in a manner that minimizes adverse effects on particles (for example, bubbles or spheres) that may be included in the contrast medium.

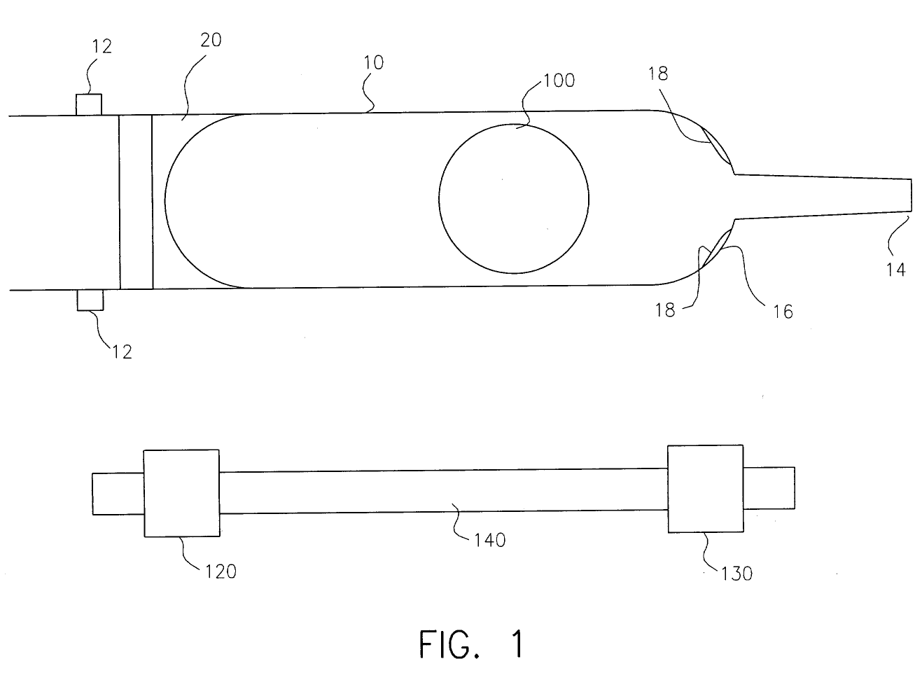

FIG. 1 illustrates one aspect of the present invention wherein a syringe 10 includes a plunger 20 slidably disposed therein for pressurizing an injection fluid within syringe 10. Also disposed within syringe 10 is preferably at least one agitation element such as a sphere or ball 100. In certain situations, generally spherical agitation elements, such as ball 100, have the advantage (as compared to agitation elements of other shapes) of symmetry regardless of orientation. Ball 100 is preferably movable within syringe 10 to cause agitation of the injection fluid within syringe 10.

As used herein to describe the present invention, the term “rearward” refers generally to a direction (along the longitudinal axis of syringe 10) toward the end of syringe 10 opposite syringe tip or outlet 14. The term “forward” refers generally to a direction toward syringe tip 14.

In one aspect of the present invention, ball 100 is moved within syringe 10 by magnets 120 and 130 that are disposed at the front and rear of syringe 10, respectively. Magnets, 120 and 130 can, for example, be electromagnets that are alternately energized to move ball 100 in a forward and rearward direction. The strength of the magnetic fields of magnets 120 and 130, the length of time each magnet is energized, the density of ball 100, the size of ball 100 and the surface structure of ball 100 can, for example, be used to control the movement of ball 100 and thereby the currents produced by ball 100 with syringe 10.

In an alternative aspect, a single magnet such as magnet 120 or magnet 130 can be moved relative to syringe 10 to create motion of ball 100. For example, magnet 120 can be moved in a forward and rearward direction along a linear track 140. The manner and speed of the movement of magnet 120 controls the motion of ball 100. In this embodiment, magnet 120 can, for example, be a permanent magnet.

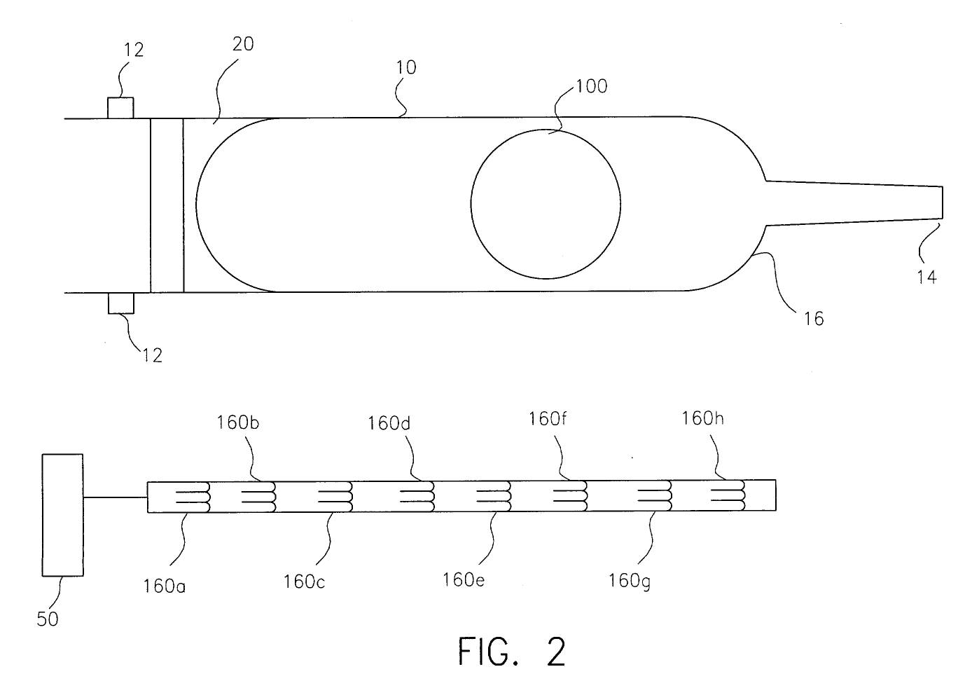

In another aspect illustrated in FIG. 2, a series of electromagnets 160a through 160h can be positioned along the length of syringe 10. Electromagnets 160a through 160h can, for example, be actuated in series to move ball 100 forward and rearward within syringe 10.

The density of ball 100 relative to the density of the fluid within syringe 10 can also affect the movement of ball 100 within syringe 10 as a result of gravitational force on ball 100. If it is desirable to remove the effects of gravity from the movement of ball 100, the density of ball 100 can be generally matched to that of the fluid. In that manner, the effects of gravity are removed and any motion of ball 100 will be a result of an applied magnetic field.

In addition to the use of magnetic fields to effect controlled motion of agitation elements within syringe 10, however, the force of gravity can also be used. In the case that gravity is used to effect motion of, for example, ball 100, the density of ball 100 or other agitation element(s) is preferablydifferent than the density of the fluid within syringe 10.

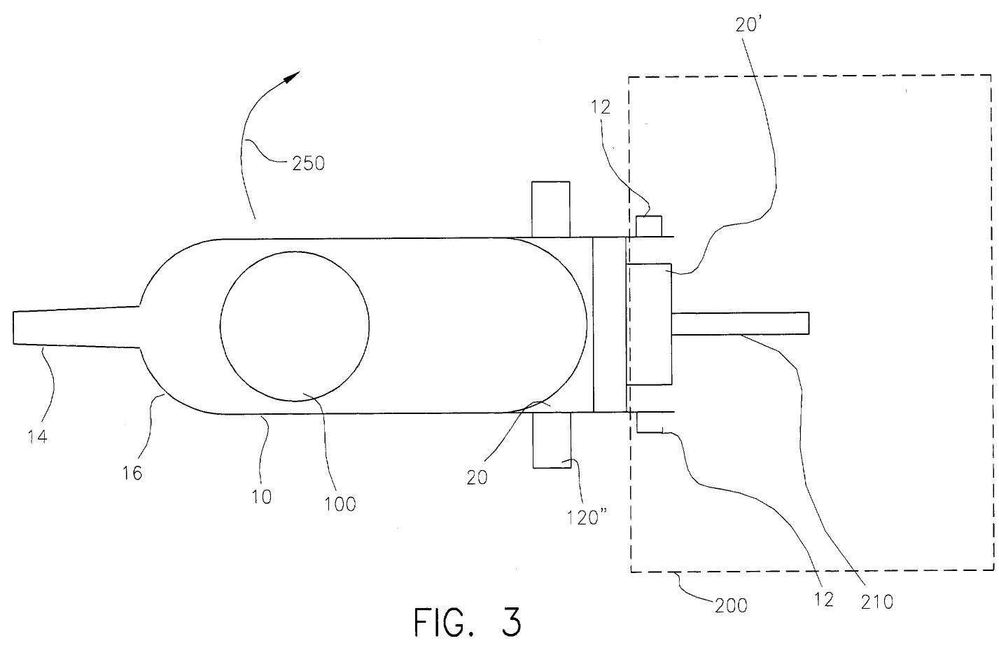

FIG. 3 illustrates an embodiment of the present invention in which both the force of gravity and a magnetic field are used to control the motion of ball 100 within syringe 10. In this embodiment, syringe 10 is attached to an injector 200 via, for example, flanges 12 that can cooperate with slots and retaining elements (not shown) in the front wall of injector 200 as known in the art. Injector 200 includes a drive member 210 such as a piston that cooperates with plunger 20 to impart a generally linear sliding motion to plunger 20 within syringe 10.

Typically, syringe 10 is preferably oriented in a downward direction during an injection procedure to cause any air bubbles within syringe 10 to travel to the rear of syringe 10 and prevent injection thereof into the patient. This downward orientation of syringe 10 is illustrated in FIG. 3. Because ball 100 has a different density than the fluid within syringe 10, gravity will cause ball 100 to move either downward (in the case that ball 100 has a greater density than the fluid) or upward (in the case that ball 100 has a density less than the fluid). In FIG. 3, ball 100 preferably has a density that is greater than the fluid. Gravity will thus tend to draw ball 100 downward through the fluid toward syringe tip 14. A magnet 120′ can, for example, be provided on the axis of drive member 210 to attract ball 100 toward the rear of syringe 10 (that is, upward in the orientation of syringe 10 in FIG. 3). Alternatively, a magnet 120″ can be positioned around the circumference of syringe 10 near a rearward end thereof. During operation of the embodiment of FIG. 3, gravity can be used to first draw ball 100 downward. Magnet 120′ or magnet 120″) is then actuated to draw ball 100 upward. When magnet 120′ is deactivated, ball 100 will once again be drawn downward by gravity. This process can be repeated to effect agitation of the fluid within syringe 10.

Additionally, syringe 10 itself can be moved to assist in agitating the fluid therein. Changing the orientation of syringe 10, for example, causes motion of particles (for example, bubbles) therein as a result of the difference of the density of those particles and the density of the fluid in which the particles are suspended. Moreover, movement of syringe 10 to change its orientation with respect to the line of gravity can also cause motion of ball 100 therein as a result of a difference between the density of ball 100 and the fluid. Syringe 100 can, for example, be rotated as indicated by arrow 250 in FIG. 3.

Multiple agitation elements such as ball 100 can be used in the present invention. In the case that syringe 10 is rotated or otherwise moved as described above, for example, it may be desirable to use at least two agitation elements or balls 100. One agitation element can, for example, have a density that is less than the density of the fluid, while the other agitation element can have a density that is greater than the density of the fluid. Likewise, multiple agitation element can be moved through the fluid within the syringe through use of one or more magnets.

In the embodiments of FIGS. 1 through 3, the shape of syringe 10 has been modified from the general shape of currently available syringes to optimize mixing and minimize wastage of contrast fluid. In that regard, transition region 16 at a forward end of syringe 10 (wherein the diameter of syringe 10 decreases to connect the barrel thereof to the neck thereof) is rounded to allow ball 100 to move far forward within syringe 10. Syringe 10 can also include ribs 18 (see FIG. 1) at a forward end of syringe 10 to prevent ball 100 from being wedged within the transition region of syringe 10.

Likewise, the shape of plunger 20 has also been modified from the general shape of currently available syringe plungers to optimize mixing and minimize wastage of contrast fluid. In that regard, plunger 20 preferably has a concave surface to allow ball 100 to move rearward within syringe 10. In general, the rounded shapes of transition region 16 and the concave surface of plunger 20 maximize the volume through which ball 100 may move in syringe 10, thereby minimizing areas of little induced flow, and assist in preventing ball 100 from becoming stuck in areas of limited space.

As discussed above, the particles within ultrasound contrast media are fragile. Although it is desirable to induce mixing within such contrast media to maintain a homogeneous concentration of particles, care should be taken to prevent destruction of the particles via creation of excessive forces thereon.

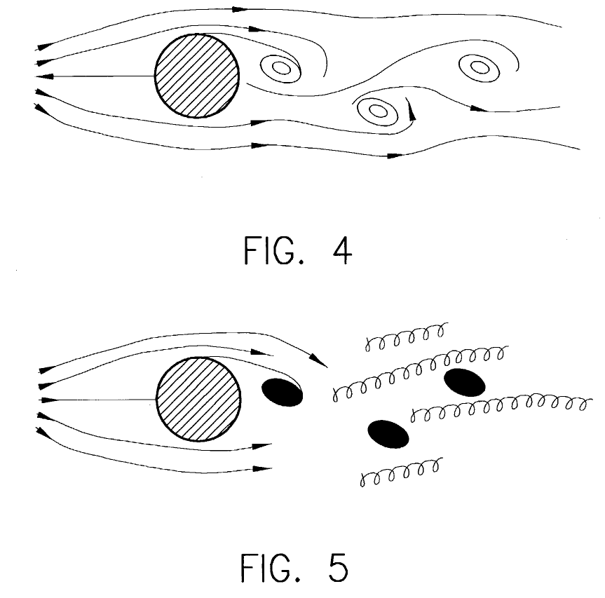

As the speed of flow increases, a more or less irregular “eddying” motion, or a state of commotion and agitation develops. This eddying motion is a result of velocity fluctuations superimposed on the main flow, within boundary layers, and within the wake behind solid bodies. At low Reynold's number values, the flow passing by a sphere within the fluid is generally steady along streamlines. Once the Reynold's number exceeds unity, however, a small amount of circulation occurs. At Reynold's number values of about 50 to 100, vortices break off and travel with the fluid and new vortices are formed behind the object. This continuous stream of vortices is termed “Kármán vortex streets” and is generally a condition that is desirable for optimal mixing of ultrasound contrast agents. An example of a flow profile including Kármán vortex streets is shown in FIG. 4.

At higher flow velocities, the Reynold's number increases further and the vorticity forms a band of irregular, chaotic flow, called the boundary layer, around the sphere as shown in FIG. 5. The increase in turbulent flow with an agitated boundary layer can cause damage to some of the microbubbles typically used in ultrasound contrast media, as a result of pressure gradients and sheer forces (resulting from relatively large velocity changes over short distances) within the suspension.

For a single spherical object having a diameter of 1 cm flowing in a fluid having a density of 1 g/cm³ and a dynamic viscosity of 6.9×10⁻² poise, the required velocity of the object for a given Reynold's number can be determined by the following equation:

$$v = \frac{η Re}{{D}_{ρ}}$$

wherein: η is the dynamic viscosity; D is the diameter of the spherical object; v is the dynamic viscosity; and Re is the Reynold's number.

For Kármán vortex street conditions of mixing (50<Re<100), to the above equation provides an object velocity range of 3.5 to 6.9 cm/sec. The force required to move the object through the liquid, in addition to the force of gravity (assuming the container is vertically oriented) can be approximated for low values of the Reynolds number (<1000) using the following equation:

F_{D}=3πμD

wherein F_{d} is the approximate drag force from the fluid flow. In the above example, F_{d}=4.87×10⁻⁵N.

A number of factors other than, fluid viscosity, element velocity and element diameter also influence the fluid flow, including: temperature, the distance between the moving object and the container wall, the compressibility and homogeneity of the of the fluid medium and the surface characteristics of the object. Surface projections, roughness, irregularities and/or imperfections in body geometry/motion generally cause eddying and mixing in the fluid in which the body is moving more readily than if the surface were regular or smooth. Surface irregularity or roughness effects become more pronounced at higher velocities.

Preferably, the diameter, velocity and surface features of the agitating elements of the present invention are selected to achieve improved (and, preferably, generally optimal) mixing of the contrast agent without damaging the agent from pressure gradients and sheer forces that may be present with increased levels of turbulence. so In general, it is preferred that the flow conditions create eddying such as Kármán vortex streets. Surface irregularities or roughness can provide such flow characteristics at lower flow velocities as compared to smooth surfaces.

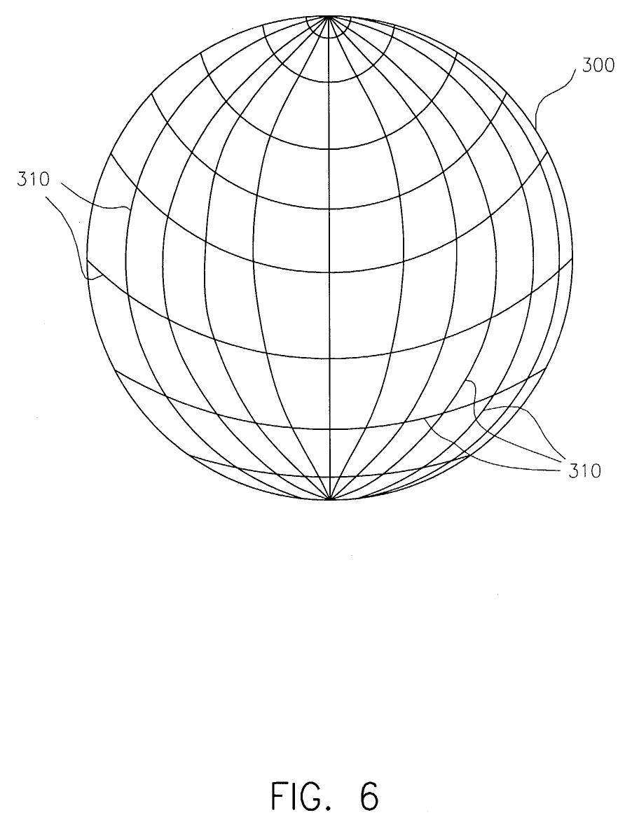

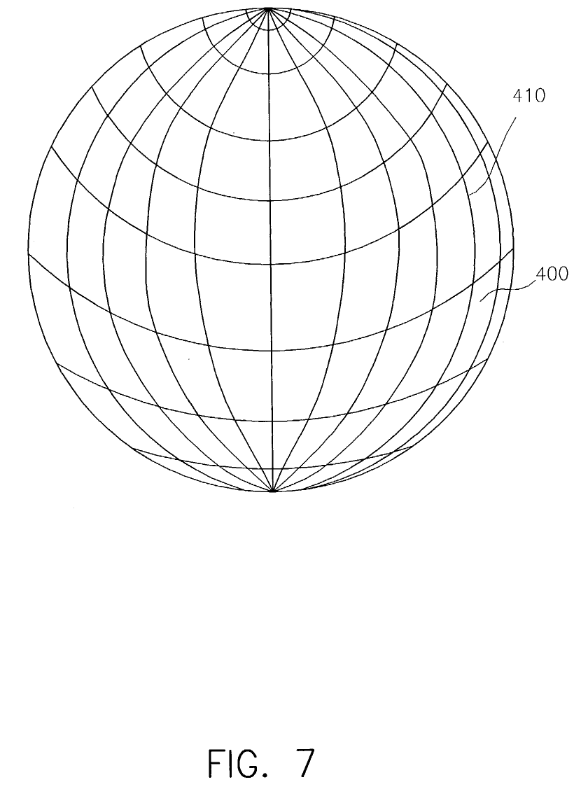

For example, as illustrated in FIG. 6, a sphere 300 can be provided with surface irregularities such as channels 310 to create vortices and mixing around sphere 300 to assist in agitation. Likewise, a mesh 410 can be overlain upon a sphere 400 to create vortices and mixing around sphere 400 as illustrated in FIG. 7. Alternatively, a “hollow” spherical mesh can be placed within the fluid. Such a spherical mesh can be envisioned by considering sphere 400 in FIG. 7 to simply represent space within spherical mesh or wire frame 410. Once again, Mixing, eddying flow, and drag are dependent on the shape of the object. In general, the greater the amount of projections or other surface features an object has, the more these protrusions disrupt the fluid surrounding it. Likewise, if the object has one or more voids, mixing flow is created. Once again, surface irregularities in general allow improved mixing at decreased object movement rates.

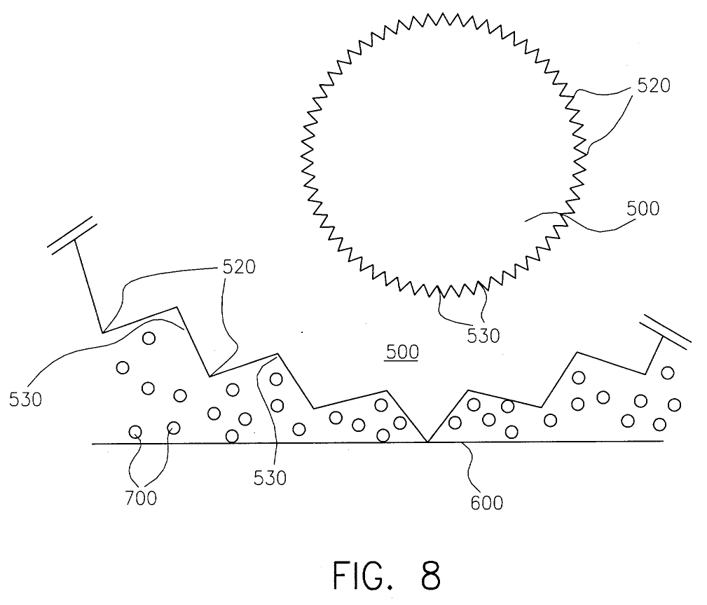

In addition to adjusting the flow field around the agitation element, surface properties of the agitation element can be designed to prevent destruction of particles with the injection fluid. For example, in the embodiment of FIG. 8, a generally spherical agitation element 500 is provided with projections 520 that define channels 530 therebetween. In general, protrusions 520 act to reduce or minimize the contact of agitation element 500 with interior wall 600 of the syringe (or with another agitation element) containing agitation element 500, thereby reducing or minimizing destruction of particles/bubbles 700 caught between agitation element 500 and syringe wall 600 (or between agitation element 500 and another agitation element).

All of the above agitation mechanisms have been discussed primarily in relation to agitation of an ultrasound contrast medium once it has been prepared. For many contrast mediums, such preparation includes mixing a powder with a liquid and vigorously mixing or agitating the mixture to create a suspension of the small particles (bubbles or solids) in a liquid which serve to scatter ultrasound energy. All of the above embodiments of the present invention are also applicable to provide injector-based initial mixing of the contrast medium. It may, however, be desirable to more vigorously mix the contrast medium to initially create a suspension that to maintain such a suspension. In that regard, the agitation devices of the present invention are easily operable at two or more levels of agitation. For example, a first, more vigorous level of agitation can be used in initial preparation of a medium. A second, less vigorous level of agitation can be used to maintain a suspension or mixing within the medium. The level of agitation and other aspects of the agitation mechanisms of the present invention are easily controlled, for example, via a controller such as controller 50 as illustrated in FIG. 2. Such a controller may, for example, include a microprocessor that can be used to adjust the frequency of actuation of electromagnets 160a through 160h to control the speed at which ball 100 moves through the fluid.

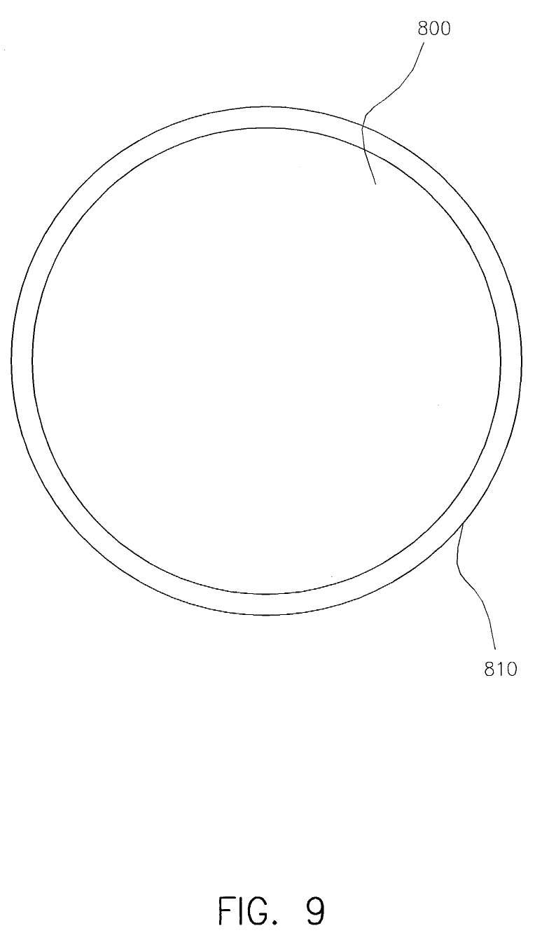

Elements within an injection fluid can also, for example, be used as a carrier in creating a multi-component injection fluid. In that regard, FIG. 9 illustrates a spherical element 800 that has been coated with a powder 810. Such a powder can also, for example, be coated within channels 310 of sphere 300 in FIG. 6 or within the void interior space of generally spherical mesh (or wire frame) element 410. Use of a coated element within a syringe facilitates the creation and subsequent agitation of a multi-component injection fluid. Likewise, use of a coated agitation element can easily be used to control the timing of release of contrast particles and/or a therapeutic drug into the injection fluid.

An element can be coated as described above via a number of processes, including, for example, spraying, rolling, dipping, evaporating or other processes, that accumulate material in layers onto the surface of the element. Time release can, for example, be effected by layering the material or interspersing layers with material that requires additional time to dissolve, thereby delaying dispersion. As an alternative, the object or element can be coated with conventional time release particles that contain coatings of various thickness, commonly used, for example, in oral medications. In all of these cases, the degree of agitation affects the release of the agent from the element. In particular, the greater the degree of agitation, the faster the release of the agent from the element.

Although the present invention has been described in detail in connection with the above embodiments and/or examples, it is to be understood that such detail is solely for that purpose and that variations can be made by those skilled in the art without departing from the spirit of the invention except as it may be limited by the following claims.

What is claimed is:

1. A system for injecting a multi-component fluid into a patient comprising: a syringe and at least one agitation element moveable within the syringe to agitate the fluid, the agitation element including surface structures to create mixing in the vicinity of the agitation element.

2. The system of claim 1 wherein the agitation element is adapted to be moved by magnetic force.

3. The system of claim 1 wherein the agitation element is generally spherical in shape with channels formed therein.

4. The system of claim 1 wherein the agitation element includes a base that is generally spherical in shape, the base having a mesh overlain thereon.

5. The system of claim 1 wherein the agitation element is a generally hollow mesh.

6. The system of claim 5 wherein agitation element is generally spherical in shape.

7. The system of claim 1 wherein the size of the agitating element, the size of the surface structures and the velocity with which the agitating element is moved within the fluid are adapted to create vortices within the fluid without creating turbulent flow of a magnitude to damage a significant number of ultrasound scattering particles disposed within the fluid.

8. A system for injecting a multi-component fluid into a patient comprising: a syringe and at least one agitation element moveable within the syringe to agitate the fluid, the agitation element including surface structures to reduce the area of contact between the agitation element and another surface.

9. The system of claim 8 wherein the agitation element includes projections extending from the surface thereof.

10. The system of claim 9 wherein the agitation element is generally spherical in shape.

11. A system for injecting a multi-component fluid into a patient comprising: a syringe and at least one agitation element moveable within the syringe to agitate the fluid, the agitation element including a coating that includes at least one component of the multi-component fluid, the coating releasing the component into the fluid.

12. The system of claim 11 wherein the component in the coating is a powder adapted to disperse particles within the fluid.

13. The system of claim 12 wherein the particles are bubbles or microspheres.

14. The system of claim 13 wherein the coating is adapted to release a therapeutic drug into the fluid.

15. A system for injecting a multi-component fluid into a patient comprising: a syringe and at least two agitating elements moveable within the syringe to agitate the fluid, a first one of the agitating elements having a density greater than a density of the fluid and a second on of the agitating elements having a density less than the density of the fluid.

16. The system of claim 15 further including a mechanism to impart motion to the syringe to change the orientation of the syringe relative to the orientation of gravitational force.
